(19) United States
(12) Patent Application Publication
(10) Pub. No.: US 2016/0135758 A1
(43) Pub. Date: May 19, 2016

(54) Patient Care Device with Audible Alarm Sensing and Backup

(71) Applicant: Segars California Partners, LP, Austin, TX (US)

(72) Inventor: Peter D. Sabota, Austin, TX (US)

(73) Assignee: Segars California Partners, LP, Austin, TX (US)

(21) Appl. No.: 14/900,154

(22) PCT Filed: Jun. 14, 2014
(86) PCT No.: PCT/US14/42439

§ 371 Date: Dec. 19, 2015

Related U.S. Application Data
(60) Provisional application No. 61/835,524, filed on Jun. 14, 2013.

(51) Int. Cl. A61B 5/00

(52) U.S. Cl. CPC A61B 5/746; A61B 5/7405

(57) ABSTRACT

An alarm sensing and backup system for patient devices that senses when the normal alarm sensing system is not sounding properly and sounds an independent backup alarm.

### CROSS-REFERENCE TO RELATED APPLICATIONS

This application claims the benefit of U.S. provisional application 61/835,524 filed Jun. 14, 2013.

### BACKGROUND

This disclosure relates to the field of various patient care devices used in the care of critical patients. Example devices could be infant warming devices, anesthesia machines, infusion pumps, ventilators, monitoring devices, or neonatal intensive care units. These patient care devices are often in a hospital or medical setting. Such patient care devices often have audible alarm condition sensors that monitor certain parameters and determine when an alarm condition exists. This alarm condition may be associated with the patient or with the patient care device itself. This could be related to a condition of the patient (eg: low heart rate, low temperature, etc.) or that there has been a critical failure in the hardware or software operating the medical device. The actual alarm sound may be distinctive to the critical condition being sensed. These alarms are very important to patient safety in that the caregivers must respond quickly to satisfy the alarm condition. A loss of hardware or system faults could go undetected and the solution described in this disclosure ensures that this type of failure will not occur.

There is a need then for a system that ensures that an alarm actually sounds when an alarm condition is detected, and that it is the correct alarm for the condition sensed.

Although the solution proposed here can be applied in any number of patient care devices the example to be shown is for an infant warming device. A variety of various infant warming devices are used to provide heat support to premature infants who cannot sustain their own body temperature. In the treatment of infants, and particularly those born prematurely, it is necessary to provide heat to the infant during the care and treatment of the infant and to minimize heat loss from the infant's body. An apparatus for providing such heat will be referred to in this disclosure as an infant warming device. In general such an apparatus comprises a flat planar surface on which the infant rests while various procedures are carried out. There are normally protective guards that surround the infant and some type of overhead heater directing radiant energy toward the infant. It should be understood that these infant warming devices might have other descriptive names, such as, for example, an infant care device, or an infant care center, patient care center, an infant incubator, or a combination device, and this disclosure anticipates any of those other names. This disclosure will use the term infant warming device.

### SUMMARY

This need can be met by incorporating into the device a separate sound-sensing device that that is part of an alarm verification system and will serve as an independent means of assuring that an alarm actually sounds and sounds correctly when it is enabled. This sound-sensing device is part of the audible alarm system circuitry and is programmed to sense or listen for the correct sound of the alarm when the audible alarm sensing system is triggered and if the correct alarm sound is not detected it will sound an independent backup alarm thus ensuring that the critical patient condition or the device fault condition is known. Sensing whether the correct sound could mean not only whether the alarm sounds, but if it loud enough or if the correct sound pattern is heard. As a secondary benefit, in an Infant warming device, this sound-sensing device can also be used to measure and/or display ambient noise surrounding the device that may be displayed on a user interface of the device.

The need can be met by a patient care device; including at least: an audible alarm sensor system to detect critical conditions of the patient or of the patient care device; one or more audible alarms that are triggered to sound by the audible alarm sensor system; an independent backup alarm for the patient care device; an alarm verification system comprising a sound sensing device to sense and verify whether the one or more audible alarms have actually sounded; wherein if the sound sensing device does not detect a correct audible alarm the alarm verification system activates the independent backup alarm.

The need can also be met by a method for providing an independent backup alarm for a patient care device having an audible alarm sensor system for detecting critical conditions and one or more audible alarms including at least the steps of: sensing the one or more audible alarms; providing an independent backup alarm; and sounding the independent backup alarm when the sound sensing device does not sense the one or more audible alarms after the audible alarm sensing system has detected critical conditions of the patient or the patient care device.

### BRIEF DESCRIPTION OF THE DRAWINGS

There are disclosed in the drawings and detailed description to follow various embodiments of the solution proposed herein. It should be understood, however, that the specific embodiments given in the drawings and entailed description do not limit the disclosure. On the contrary, they provide the foundation for discerning the alternative forms, equivalents, and modifications that will be encompassed in the scope of the eventual claims.

FIG. 1 is a view of an infant care center that can include the inventive concept described in this disclosure.

FIG. 2 is an alternate view of an infant care center that can include the inventive concept described in this disclosure.

FIG. 3 is an alternate view of an infant care center that can include the inventive concept described in this disclosure.

FIG. 4 is a view illustrating the location of the alarms and the sound-sensing device of the alarm verification system above the integrated touch screen.

FIG. 5 is a cutaway view illustrating the alarms and the sound-sensing device of the alarm verification system above the integrated touch screen.

### DETAILED DESCRIPTION

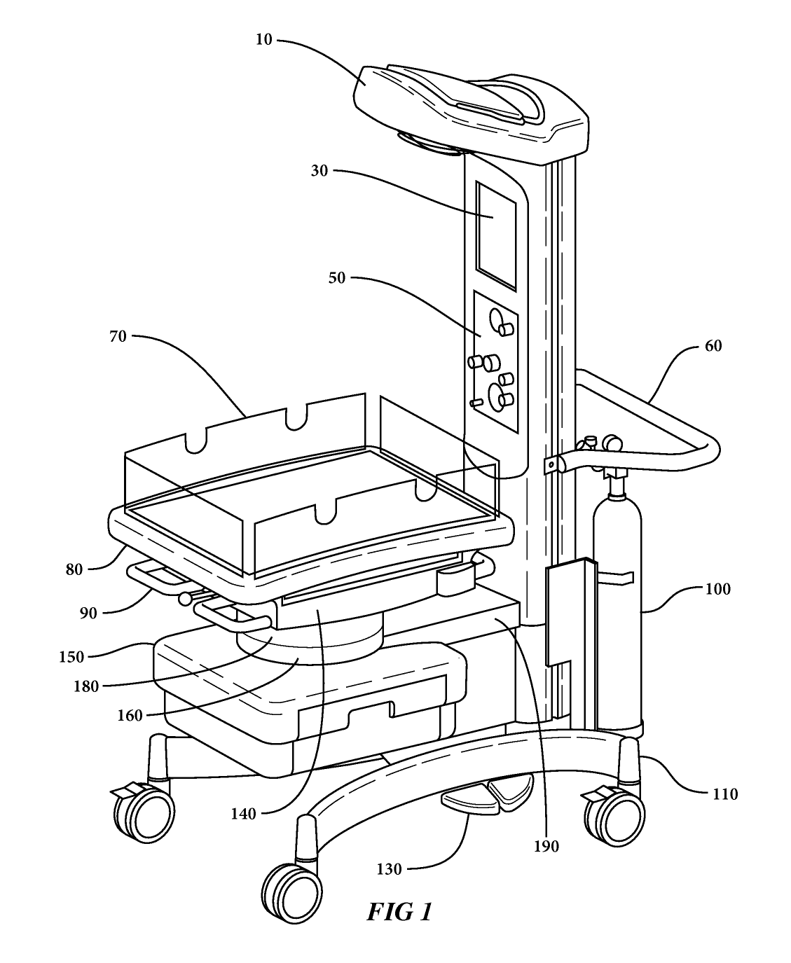

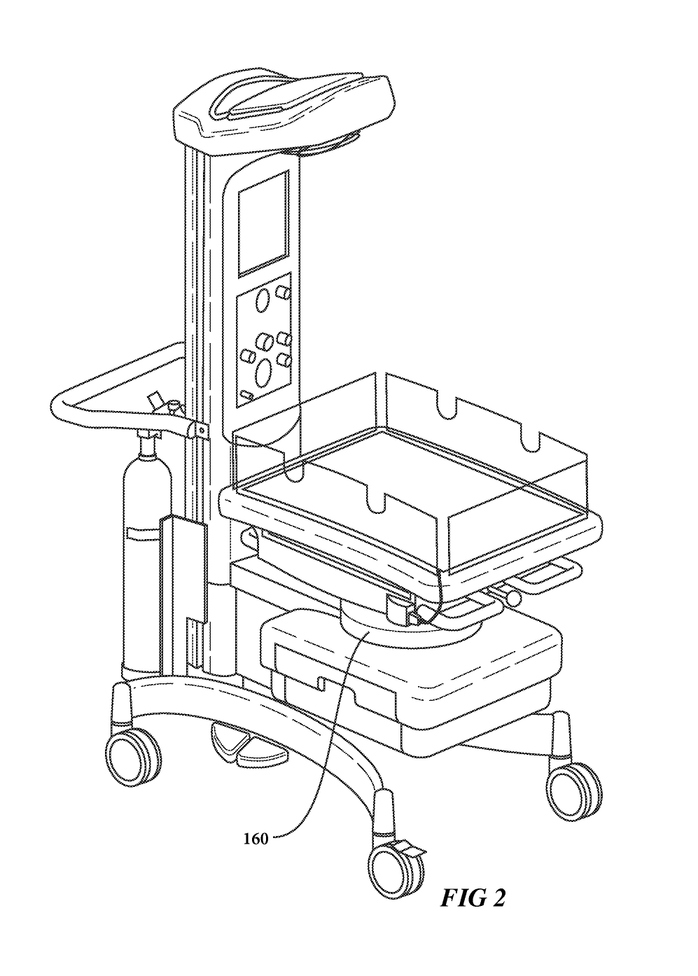

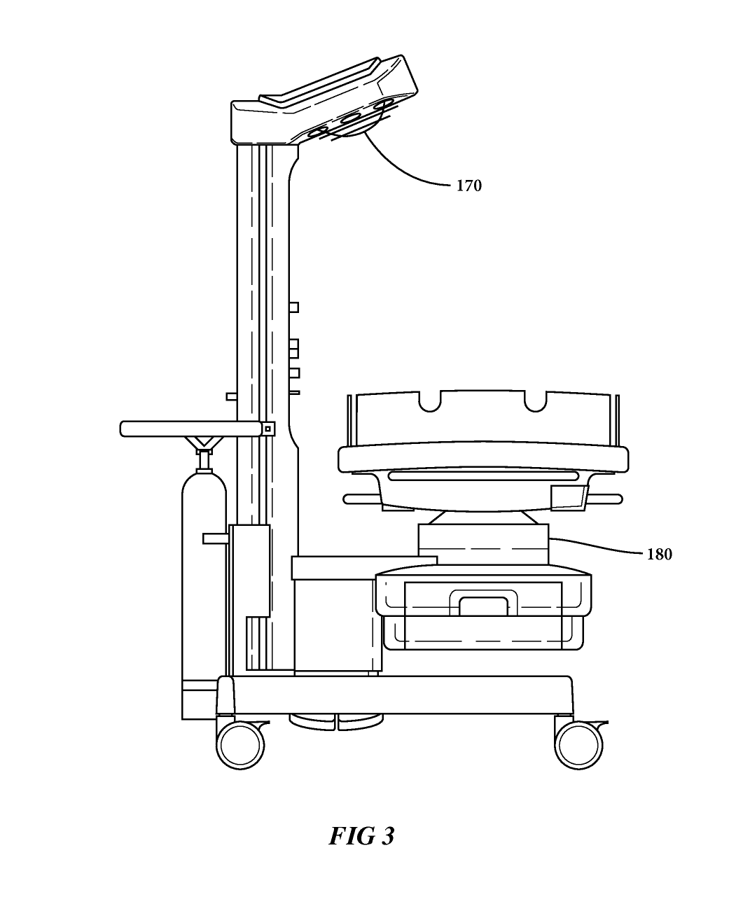

Referring now to FIGS. 1, 2, and 3, several views of an infant warming center that can include the inventive concept to be described in this disclosure. The center includes an infant bed 80 that underlies an infant positioned thereon. The infant bed has a surrounding sidewall 70 and rides upon a patient support mechanism 140. The patient bed and surrounding sidewalls may enclose a heated mattress. A vertical column structure mounted on the infant warming center supports a radiant heater head 10, containing a radiant heater 170 (FIG. 3), with that radiant heater. The radiant heater assembly is designed to optimize the heat focused on the infant. The vertical column structure may have a user interface/display 30 which can act as a display for the images recorded by the camera. The column may include a resuscitation module 50. The infant warming device's main computer controller may reside in the vertical column structure or may reside in the patient support mechanism. Handles 60, 90, are used to move the infant warming device around as it can be moved on flat surfaces via legs 110 with attached wheels and controlled with footswitches 130. On the rear side of the column is a location for carrying a remote gas supply tank 100. Under the patient support mechanism 140 is a cantilever cover 160 and turret cover 180 for shrouding the rotation mechanisms, with a cantilever arm 190 that supports the patient support, vertical column, and supports a storage enclosure 150.

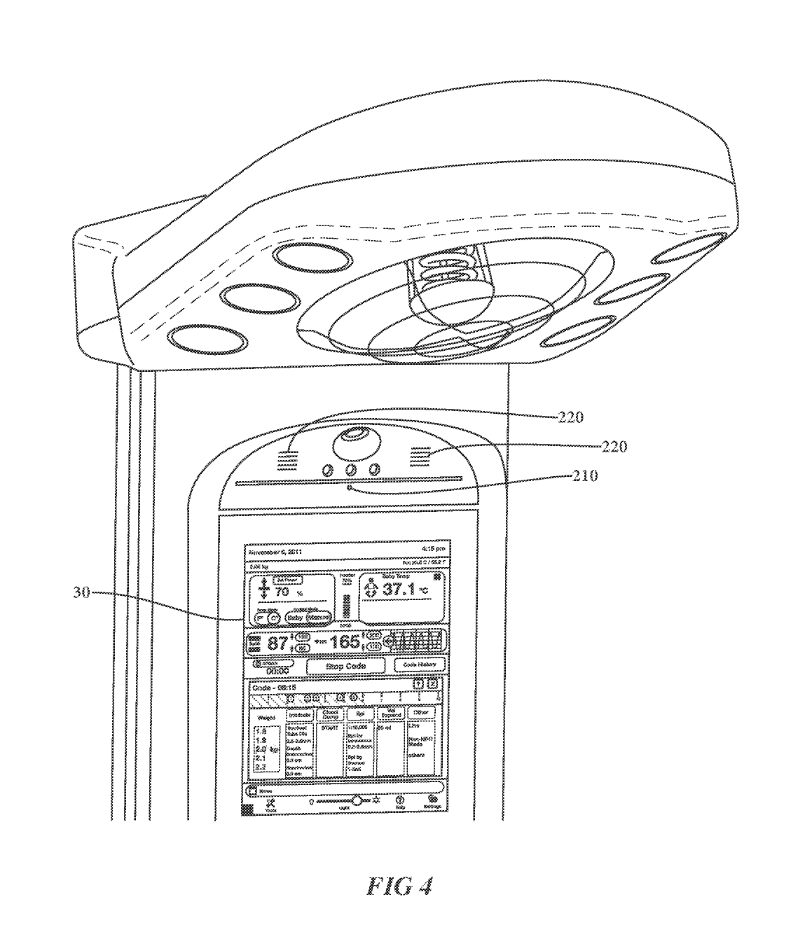

FIG. 4 is a view illustrating a possible location of the alarms 220 of an audible alarm sensing system and the sound sensing device 210 of an alarm verification system above the integrated touch screen 30. There may be one or more alarms 220. The sound-sensing device, which may be a microphone, is located in this illustration on the vertical column structure below alarms 220 of the normal audible alarm sensing system. This sound sensing device of the alarm verification system will measure the sound of the alarm after the audible alarm sensing system is triggered and if the correct alarm is not detected or is too low in volume it will sound an independent backup alarm (not shown) thus ensuring that the critical patient condition or the device fault condition is known. In addition, there may be different types of alarms—different sound patterns—sounded by the audible alarm sensing system to signify different critical issues to be addressed in either the equipment or the patient. Example alarm patterns from medical alarm standards are described in terms of Pulse, Pulse Frequency, Harmonic Components, Pulse Duration, Rise/Fall Time, Tempo, and Pulse Sound Pressure (volume). Another alarm pattern can be a voice alarm.

The proposed system may be programed to detect for the presence of any of these alternate patterns. The proposed alarm verification system may also be able to sense and verify whether the correct alarm is sounded and if not, sound the independent backup alarm.

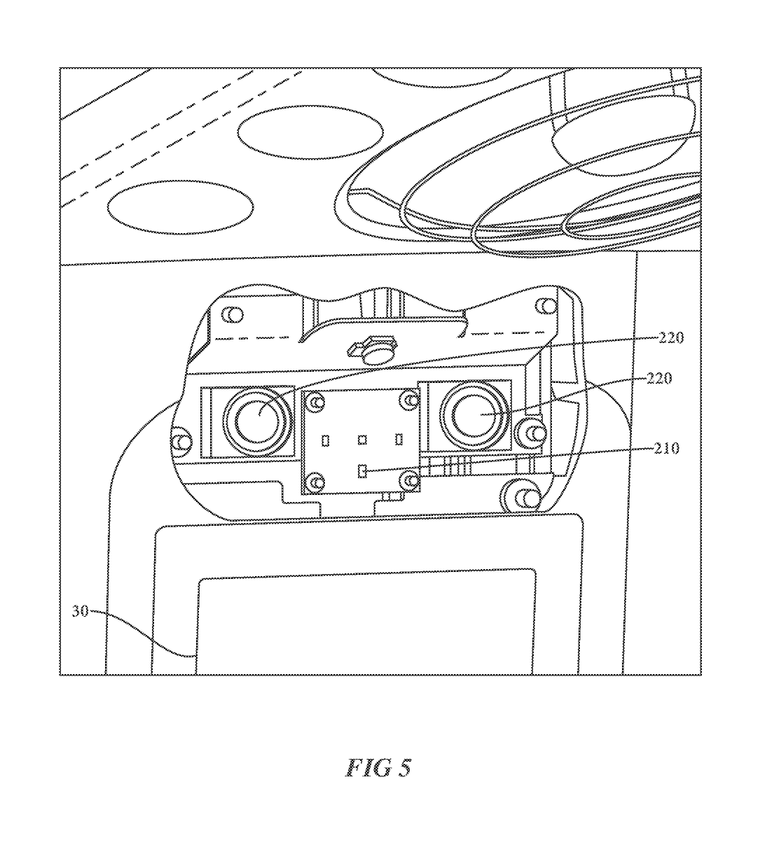

FIG. 5 is a cutaway view illustrating the alarms 220 of the audible alarm sensing system and the sound-sensing device 210 of the alarm verification system above the integrated touch screen 30. The sound-sensing device is shown mounted on a circuit board but the deployment is not limited to that approach. There may be one or more alarms 220. Alarms 220 are shown in this illustration on each side and slightly above the sound-sensing device 210. The exact placement is not critical.

In practice the included sound-sensing device 210 either constantly monitors for sound or only monitors when the audible alarm sensing system is triggered and as part of the circuitry of the alarm system checks for the alarm sounds to come from alarms 220 in an alarm situation. If it does not detect the alarm signal or the sound is below a predefined threshold volume it automatically sounds an independent backup alarm.

In the proposed audible alarm sensing system, the sound sensing device is programmed to “sense” the normal audible alarm whenever the audible alarm sensing system is triggered by any undesired condition.

### Advantages of the Invention Over the Prior Art

The alarm verification system described herein increases the level of safety for the patient by adding significant reliability to the alarm system.

Although certain embodiments and their advantages have been described herein in detail, it should be understood that various changes, substitutions and alterations could be made without departing from the coverage as defined by the appended claims. Moreover, the potential applications of the disclosed techniques is not intended to be limited to the particular embodiments of the processes, machines, manufactures, means, methods and steps described herein. As a person of ordinary skill in the art will readily appreciate from this disclosure, other processes, machines, manufactures, means, methods, or steps, presently existing or later to be developed that perform substantially the same function or achieve substantially the same result as the corresponding embodiments described herein may be utilized. Accordingly, the appended claims are intended to include within their scope such processes, machines, manufactures, means, methods or steps.

What is claimed is:

1. A patient care device; comprising:
a. an audible alarm sensor system to detect critical conditions of the patient or of the patient care device;
b. one or more audible alarms that are triggered to sound by the audible alarm sensor system;
c. an independent backup alarm for the patient care device;
d. an alarm verification system comprising a sound sensing device to sense and verify whether the one or more audible alarms have actually sounded;
e. wherein if the sound sensing device does not detect a correct audible alarm the alarm verification system activates the independent backup alarm.

2. The patient care device of claim 1 wherein the patient care device is an infant warming center.

3. The patient care device of claim 2 further comprising a vertical column structure mounted on the infant warming center that supports a radiant heater head, containing a radiant heater assembly that is one of the associated warming mechanisms.

4. The patient care device of claim 3 wherein the sound sensing device to sense the audible alarm and the independent backup alarm are mounted within the patient care device.

5. The patient care device of claim 4 wherein the sound sensing device to sense the audible alarm and the independent backup alarm are mounted in the vertical column structure.

6. The patient care device of claim 1 wherein the patient care device is an anesthesia machine.

7. The patient care device of claim 1 wherein the patient care device is an infusion pump.

8. The patient care device of claim 1 wherein the patient care device is a ventilator.

9. The patient care device of claim 1 wherein the patient care device is a monitoring device.

10. The patient care device of claim 1 wherein the patient care device is in a hospital or medical setting.

11. The patient care device of claim 1 wherein the one or more audible alarms can vary in pattern for differing critical conditions.

12. The patient care device of claim 11 wherein the sound-sensing device can sense and verify whether a correct alarm pattern is sounded for each differing critical condition.

13. The patient care device of claim 12 wherein the sound-sensing device can sense and verify whether the correct alarm pattern is sounded with sufficient volume.

14. The patient care device of claim 12 wherein the sound-sensing device can sense and distinguish at least between correct alarm patterns based on Pulse, Pulse Frequency, Harmonic Components, Pulse Duration, Rise/Fall Time, or Tempo.

15. The patient care device of claim 12 wherein the sound-sensing device can sense a voice alarm.

16. The patient care device of claim 1 wherein the sound sensing device is a microphone.

17. The patient care device of claim 1 wherein the one or more audible alarms are sounded by speakers.

18. A method for providing an independent backup alarm for a patient care device having an audible alarm sensor system for detecting critical conditions and one or more audible alarms comprising:
a. sensing the one or more audible alarms;
b. providing an independent backup alarm; and
c. sounding the independent backup alarm when the sound sensing device does not sense the one or more audible alarms after the audible alarm sensing system has detected critical conditions of the patient or the patient care device.

19. The method for providing an independent backup alarm for a patient care device having an audible alarm sensor system for detecting critical conditions and one or more audible alarms of claim 18 wherein the one or more audible alarms can vary in pattern for differing critical conditions.

20. The method for providing an independent backup alarm for a patient care device having an audible alarm sensor system for detecting critical conditions and one or more audible alarms of claim 19 wherein the sensing of the one or more audible alarms can sense and distinguish at least between correct alarm patterns based on Pulse, Pulse Frequency, Harmonic Components, Pulse Duration, Rise/Fall Time, or Tempo.

21. The method for providing an independent backup alarm for a patient care device having an audible alarm sensor system for detecting critical conditions and one or more audible alarms of claim 19 wherein the sensing of the one or more audible alarms can sense voice alarms.

22. The method for providing an independent backup alarm for a patient care device having an audible alarm sensor system for detecting critical conditions and one or more audible alarms of claim 19 wherein the sensing of the one or more audible alarms can sense and verify whether the correct alarm pattern is sounded for each differing critical condition.

23. The method for providing an independent backup alarm for a patient care device having an audible alarm sensor system for detecting critical conditions and one or more audible alarms of claim 19 wherein the sensing of the one or more audible alarms can sense and verify whether the correct alarm pattern is sounded with sufficient volume.

24. The method for providing an independent backup alarm for a patient care device having an audible alarm sensor system for detecting critical conditions and one or more audible alarms of claim 18 wherein the patient care device is an infant warming center.

25. The method for providing an independent backup alarm for a patient care device having an audible alarm sensor system for detecting critical conditions and one or more audible alarms of claim 18 further comprising a vertical column structure mounted on the infant warming center that supports a radiant heater head, containing a radiant heater assembly that is one of the associated warming mechanisms.

26. The method for providing an independent backup alarm for a patient care device having an audible alarm sensor system for detecting critical conditions and one or more audible alarms of claim 18 wherein the sensing of the one or more audible alarms and the independent backup alarm are mounted in the patient care device.

27. The method for providing an independent backup alarm for a patient care device having an audible alarm sensor system for detecting critical conditions and one or more audible alarms of claim 18 wherein the sensing of the one or more audible alarms and the independent backup alarm are mounted in the vertical column structure.

28. The method for providing an independent backup alarm for a patient care device having an audible alarm sensor system for detecting critical conditions and one or more audible alarms of claim 18 wherein the patient care device is an anesthesia machine.

29. The method for providing an independent backup alarm for a patient care device having an audible alarm sensor system for detecting critical conditions and one or more audible alarms of claim 18 wherein the patient care device is an infusion pump.

30. The method for providing an independent backup alarm for a patient care device having an audible alarm sensor system for detecting critical conditions and one or more audible alarms of claim 18 wherein the patient care device is a ventilator.

31. The method for providing an independent backup alarm for a patient care device having an audible alarm sensor system for detecting critical conditions and one or more audible alarms of claim 18 wherein the patient care device is a monitoring device.

32. The method for providing an independent backup alarm for a patient care device having an audible alarm sensor system for detecting critical conditions and one or more audible alarms of claim 18 wherein the patient care device is in a hospital or medical setting.

33. The method for providing an independent backup alarm for a patient care device having an audible alarm sensor system for detecting critical conditions and one or more audible alarms of claim 18 wherein the sensing of the one or more alarms is done by a microphone.

34. The method for providing an independent backup alarm for a patient care device having an audible alarm sensor system for detecting critical conditions and one or more audible alarms of claim 18 wherein the sounding of the independent backup alarm is done by speakers.
